(19) United States
(12) Patent Application Publication
(10) Pub. No.: US 2004/0259169 A1
(43) Pub. Date: Dec. 23, 2004

(54) Methods for selection for efficient animal growth

(76) Inventors: Lucina Galina-Pantoja, Franklin, KY (US); John Bastiaansen, Rosmalen (NL); Martha A. Mellencamp, Berkeley, CA (US)

Correspondence Address: MCKEE, VOORHEES & SEASE, P.L.C., 801 GRAND AVENUE, SUITE 3200, DES MOINES, IA, 50309-2721, US

(21) Appl. No.: 10/600,111

(22) Filed: Jun. 20, 2003

(51) Int. Cl. C12Q 1/68; G01N 33/53; G01N 33/567

(52) U.S. Cl. 435/7.2; 435/6

(57) ABSTRACT

The present invention relates generally to animal breeding methods. More specifically, the invention relates to methods for selecting for robustness among two or more animals based upon the quantity of immune cell subtypes and frequency of proliferative responses of lymphocytes in the animals.

### BACKGROUND OF THE INVENTION

1. Field of the Invention

The present invention relates generally to animal breeding methods. More specifically, the invention relates to methods for selecting for robustness among two or more animals based upon the quantity of immune cell subtypes and frequency of proliferative responses of lymphocytes in the animals.

2. Description of the Related Art

Animals have a complex array of molecular and cellular defenses, collectively referred to as the immune system, that recognize and attack potentially harmful foreign or endogenous but abnormal cells (respectively represented by, e.g., pathogens such as bacteria or viruses, and cancerous or pathogen-infected cells), but that do not attack but rather tolerate endogenous normal cells. When stimulated by foreign or abnormal biomolecules, the immune system undergoes a series of activities designed to neutralize and destroy the pathogens, or cancerous or pathogen-infected cells, with which the foreign or abnormal biomolecules are associated. These activities, collectively known as an immune response, may consist of a cell-mediated immune response, a humoral (antibody-mediated) immune response, or an immune response that includes elements of cell-mediated and humoral responses.

Humoral immune responses are mediated by antibodies, glycoproteins that bind specific foreign or abnormal biomolecules and attract other components of the immune system thereto. Antibodies are immunoglobulin (Ig) molecules produced by B cells, lymphocytes which originate in avian bursa or in mammalian bone marrow but migrate to and mature in other organs, particularly the spleen (Robertson, 1983). Cell-mediated immune responses are the result of activities of T cells, lymphocytes that undergo maturation within the thymus of an animal (Tizard, 1988).

T cell activities vary considerably among different subpopulations of T cells within an animal. Cytotoxic T cells recognize and destroy foreign cells (graft rejection) or endogenous but abnormal cells (e.g., cancerous cells or cells infected with intercellular parasites such as viruses and bacteria). Helper T cells interact with, and produce biomolecules that influence the behavior of, both B cells and cytotoxic T cells, in order to promote and direct antibody production and cytotoxic activities, respectively (Mosier, 1967). Other classes of T cells, including suppressor T cells and memory T cells, also exist (Miedema and Melief, 1983; Tizard, supra, pp. 225-8). Classes of T cells are to some extent distinguished on the basis that different T cells display different CD antigens on their surfaces.

In order to function properly, the T and B cells of an animal's immune system must accurately and reliably identify an enormous number of non-self compositions that are foreign, or endogenous but abnormal compositions. Recognition and identification by the immune system occurs at the molecular level. An antigen, a molecular composition having the potential to generate an immune response, is composed of one or more molecular-sized identifying features known as epitopes. A polypeptide antigen which has an amino acid sequence which comprises, for example, a hundred amino acids might comprise dozens of epitopes, wherein each epitope is defined by a portion of the polypeptide comprising from about 3 to about 15 amino acids. The number of eptitopes derivable from polypeptides alone is estimated to be about ten million (Tizard, supra, p. 25).

An antigen encountered by a T or B cell of an animal must be identified as either being associated with normal endogenous (i.e., self) antigens, an immune response to which would be injurious to the animal, or with foreign or abnormal (i.e., non-self) antigens, to which an immune response should be mounted. The process can be analogized to “friend or foe” identification in human combat. If the immune system fails to identify antigens associated with invading pathogens or tumor cells as non-self, then these “enemies” can slip through the system's defenses. If the immune system mistakenly identifies an animal's endogenous antigens as non-self, then the parts of the animal's body that comprise the endogenous antigens will face “friendly fire” from the immune system. The latter situation, in which an animal's immune system mistakenly wages cellular and molecular “war” against another, normal part of an animal's body, is known generally as “autoimmune disease.”

As part of the immune system's means of identifying antigens, individual T and. B cells produce antigen receptors which are displayed on the T or B cell's surface and which bind specific antigens. Although each individual T or B cell displays identical antigen receptors, an animal's collection of different antigen receptors is quite diverse. For T or B cells, binding of antigen to a cell's antigen receptor activates the cell, i.e., stimulates the cell to undertake activities related to generating a cell-mediated or humoral immune response. Although B cells can directly bind antigen, T cells respond to antigen only when it is displayed on specific classes of other cells known generically as antigen-presenting cells (APCs). APCs, e.g., macrophages and dendritic cells, present antigens derived from polypeptides via glycoproteins, known as MHC (major histocompatibility complex) proteins, which are displayed on the surface of APCs (Bevan et al., 1994). Without MHC proteins, T cells would not be able to distinguish between foreign or endogenous antigens.

Over the last decade, most livestock industries have developed estimated breeding values to allow identification of the best breeding animals. These estimated breeding values are calculated based on performance information of several traits from the individual animal and its relatives, and represent the most accurate criteria to identify animals of high genetic merit. It is widely believed, however, that environmental challenges like disease limit the expression of the “true” genetic potential of animals such as pigs. This may be due to the fact that selection occurs in high health status units that differ significantly from the environments encountered in commercial production. This degradation of health status, and concomitant reduction in the performance of the individual during multiplication, is observed in many plant and animal systems. Like other agricultural systems, today's livestock industry has a need for robust animals that perform well under different commercial settings. Consequently, there is a need to develop tools that can identify and/or sort the most suitable animals for particular production systems. Because of the importance of an animal's immune system in its overall health, it is possible that a correlation exists between an animal's robustness and the quantity of specific immune cells the animal possesses. It is similarly possible that a correlation exists between robustness and the functionality of particular immune cells.

### SUMMARY OF THE INVENTION

According to one aspect of the invention, there is provided a method for selecting for robustness among two or more animals comprising providing two or more animals of the same species; determining in each animal the quantity of CD16 antigen-expressing cells; and selecting the animal with the lowest quantity of CD16 antigen-expressing cells.

According to yet another aspect of the invention, there is provided a method for selecting for robustness among two or more animals comprising providing two or more animals of the same species; determining in each animal the quantity of CD16 and CD2 double-positive antigen-expressing cells; and selecting the animal with the lowest quantity of CD16 and CD2 double-positive antigen-expressing cells.

According to yet another aspect of the invention, there is provided a method for selecting for robustness among two or more animals comprising providing two or more animals of the same species; determining in each animal the quantity of CD8 antigen-expressing cells; and selecting the animal with the lowest quantity of CD8 antigen-expressing cells.

According to yet another aspect of the invention, there is provided a method for selecting for robustness among two or more animals comprising providing two or more animals of the same species; determining in each animal the quantity of MHC-DQ antigen-expressing cells; and selecting the animal with the highest quantity of MHC-DQ antigen-expressing cells.

According to yet another aspect of the invention, there is provided a method for selecting for robustness among two or more animals comprising providing two or more animals of the same species; determining in each animal the quantity of cells expressing an antigen that is targeted by MHC-DQ antibodies as MHC-DQB; and selecting the animal with the highest quantity of cells expressing an antigen that is targeted by MHC-DQ antibodies as MHC-DQB.

According to yet another aspect of the invention, there is provided a method for selecting for robustness among two or more animals comprising providing two or more animals of the same species; determining in each animal the quantity of cells expressing an antigen that is targeted by MHC-DQ antibodies as MHC-DQD; and selecting the animal with the highest quantity of cells expressing an antigen that is targeted by MHC-DQ antibodies as MHC-DQD.

According to yet another aspect of the invention, there is provided a method for selecting for robustness among two or more animals comprising providing two or more animals of the same species; determining in each animal the proliferation frequency of CD4 antigen-expressing cells; and selecting the animal with the lowest proliferation frequency of CD4 antigen-expressing cells.

The species in which robustness can be selected for include, but are not limited to, Bos taurus (cow), Sus scrofa (pig), Ovis aries (sheep), Bison bison (bison), Babalus babalus (buffalo), Gallus domesticus (chicken), Meleagrus gallopavo (turkey), Anas rubripes (duck), and Branta canadensis (goose).

It is contemplated that each of the methods of the invention described herein can be used alone or in combination with each other to select for the most robust animals.

### DETAILED DESCRIPTION OF THE INVENTION

The present invention provides methods for selecting for robustness among two or more animals.

Definitions

As used herein, the terms “robust” and “robustness” are intended to refer to the general condition in an animal characterized by higher than average (1) lifetime average daily gain (ADG), (2) hot carcass measurements, and (3) feed conversion.

As used herein, the term “CD” is intended to refer to cluster of differentiation. This designation is the international standard for leukocyte antigens for which monoclonal antibodies have been developed.

As used herein, the term “antibody” is intended to refer to a protein molecule synthesized by a B-cell upon exposure to antigen that is capable of combining specifically with that antigen; the term “monoclonal antibody” is intended to refer to an antibody molecule produced by a hybridoma that contains only one species of an antigen binding site capable of immunoreacting with a particular epitope of an antigen. A “primary” antibody is one that binds antigen directly. A “secondary” antibody is one that binds the primary antibody.

As used herein, the term “antigen” is intended to refer to a molecule or composition of matter which induces an immune response in an animal, and interacts specifically with antigen-recognizing components of an animal's immune system.

As used herein, the term “mitogen” is intended to refer to a compound that stimulates lymphocytes to transit through the cell cycle. A suitable mitogen for T-cells is Concanavalin A and phytohemagglutinin (PHA).

Determining Percentage of Antigen-Expressing Cells

A determination of the percentage of antigen-expressing cells, including cells that express the CD16, CD2, CD8, and MHC-DQ antigens, as well as cells expressing an antigen that is targeted by MHC-DQ antibodies as MHC-DQ bright (B) or MHC-DQ dull (D), in an animal having such cells is preferably accomplished by isolating periperhal blood mononuclear cells (PBMCs) from the animal; incubating the PBMCs with a primary monoclonal antibody specific for the antigen of interest; labeling the PBMCs with a secondary antibody conjugated to a fluorescent dye; counting the PBMCs that express the antigen of interest; and calculating the percentage of PBMCs that express the antigen of interest.

Determining Proliferation Frequency of Antigen-Expressing Cells

A determination of the proliferation frequency of antigen-expressing cells, including cells that express the CD4 antigen, in an animal having such cells is preferably accomplished by isolating periperhal blood mononuclear cells (PBMCs) from the animal; labeling the PBMCs with a suitable fluorescent dye; culturing the PBMCs with a suitable blastogenic medium; incubating the PBMCs with a mitogen; incubating the PBMCs with a monoclonal antibody specific for the antigen-expressing cell of interest; and determining the frequency with which the antigen-expressing cell of interest has proliferated.

Isolating PBMCs

PBMCs are preferably isolated by gradient separation with a lymphocyte separation media. A suitable lymphocyte separation media is LSM® (ICN Biomedicals).

Counting Antigen-Expressing Cells

In the invention, the counting of specific antigen-expressing cells, and the ultimate determination of the percentage of PBMCs that express that antigen, is preferably accomplished by flow cytometry, more specifically fluorescence flow cytometry. Generally speaking, flow cytometry consists of passing cells one at a time through a sensing zone of a flow cell. Since cells are passed through the flow cell one at a time, it is typically necessary to dilute the cell sample prior to analysis so that individual cells can be isolated for sensing.

A fluorescence flow cytometer incorporates the principles of fluorescence cell analysis with light scatter. In general, this requires that the cells be stained with an appropriate color dye, or that a fluorochrome label be covalently attached to an antigen or antibody on the cells' surface thus indicating the occurrence of a specific antigen-antibody reaction.

In fluorescence flow cytometry, a suspension of previously stained or fluorescently labeled particles, typically cells in a blood or other biological fluid sample, is transported through a flow cell where the individual particles in the sample are illuminated with one or more focused light beams. One or more detectors detect the interaction between the light beam(s) and the labeled particles flowing through the flow cell. Commonly, some of the detectors are designed to measure fluorescent emissions, while other detectors measure scatter intensity or pulse duration. Thus, each particle that passes through the flow cell can be mapped into a feature space whose axes are the emission colors, light intensities, or other properties, i.e. scatter, measured by the detectors. Preferably, the different particles in the sample can be mapped into distinct and non-overlapping regions of the feature space, allowing each particle to be analyzed based on its mapping in the feature space.

Proliferation Frequency Determination

In the invention, the determination of the frequency with which an antigen-expressing cell has proliferated is preferably accomplished by flow cytometry, as described herein. Flow cytometric proliferation frequency data analysis is preferably accomplished with a proliferation module-containing software program such as ModFit LT (Verity Software House, Inc., Topsham, Me.). Proliferation frequency determination is based upon the principle that each generation of cells should have approximately half of the dye of the parental cells.

Monoclonal Antibodies

A monoclonal antibody composition typically displays a single binding affinity for a particular protein with which it immunoreacts. A monoclonal antibody to an epitope of an antigen (i.e., CD2) can be prepared by using a technique which provides for the production of antibody molecules by continuous cell lines in culture. These include but are not limited to the hybridoma technique originally described by Kohler and Milstein, and the more recent human B cell hybridoma technique (Kozbor et al., 1983), EBV-hybridoma technique (Cole et al., 1985)), and trioma techniques. Other methods which can effectively yield monoclonal antibodies useful in the present invention include phage display techniques (Marks et al., 1992)).

Generally, Kohler and Milstein's hybridoma technique begins with immunizing an animal with a protein or a fragment thereof. The immunization is typically accomplished by administering the immunogen to an immunologically competent mammal in an immunologically effective amount. Preferably, the mammal is a rodent such as a rabbit, rat or mouse. The mammal is then maintained for a time period sufficient for the mammal to produce cells secreting antibody molecules that immunoreact with the immunogen. Such immunoreaction is detected by screening the antibody molecules so produced for immunoreactivity with a preparation of the immunogen protein. Optionally, it may be desired to screen the antibody molecules with a preparation of the protein in the form in which it is to be detected by the antibody molecules in an assay, e.g., a membrane-associated form of the antigen (i.e., CD2). These screening methods are well known to those of skill in the art. Next, a suspension of antibody-producing cells removed from each immunized mammal secreting the desired antibody is then prepared. After a sufficient time, the mammal is sacrificed and somatic antibody-producing lymphocytes are obtained. Antibody-producing cells may be derived from the lymph nodes, spleens and peripheral blood of primed animals. Spleen cells are preferred, and can be mechanically separated into individual cells in a physiologically tolerable medium using methods well known in the art. Mouse lymphocytes give a higher percentage of stable fusions with the mouse myelomas described below. Rat, rabbit, and frog somatic cells can also be used. The spleen cell chromosomes encoding desired immunoglobulins are immortalized by fusing the spleen cells with myeloma cells, generally in the presence of a fusing agent such as polyethylene glycol (PEG). Any of a number of myeloma cell lines may be used as a fusion partner according to standard techniques.

The resulting cells, which include the desired hybridomas, are then grown in a selective medium, such as HAT medium, in which unfused parental myeloma or lymphocyte cells eventually die. Only the hybridoma cells survive and can be grown under limiting dilution conditions to obtain isolated clones. The supernatants of the hybridomas are screened for the presence of antibody of the desired specificity, such as by immunoassay techniques using the antigen that has been used for immunization. Positive clones can then be subcloned under limiting dilution conditions and the monoclonal antibody produced can be isolated. Various conventional methods exist for isolation and purification of the monoclonal antibodies so as to free them from other proteins and other contaminants. Commonly used methods for purifying monoclonal antibodies include ammonium sulfate precipitation, ion exchange chromatography, and affinity chromatography (see, e.g., Zola et al., 1982). Hybridomas produced according to these methods can be propagated in vitro or in vivo (in ascites fluid) using techniques known in the art.

Generally, the individual cell line may be propagated in vitro, for example in laboratory culture vessels, and the culture medium containing high concentrations of a single specific monoclonal antibody can be harvested by decantation, filtration or centrifugation. Alternatively, the yield of monoclonal antibody can be enhanced by injecting a sample of the hybridoma into a histocompatible animal of the type used to provide the somatic and myeloma cells for the original fusion. Tumors secreting the specific monoclonal antibody produced by the fused cell hybrid develop in the injected animal. The body fluids of the animal, such as ascites fluid or serum, provide monoclonal antibodies in high concentrations.

Media and animals useful for the preparation of these compositions are both well known in the art and commercially available and include synthetic culture media, inbred mice and the like. An exemplary synthetic medium is Dulbecco's minimal essential medium (DMEM; Dulbecco et al., 1959) supplemented with 4.5 gm/l glucose, 20 mM glutamine, and 20% fetal calf serum. An exemplary inbred mouse strain is the Balb/c.

Fluorescent Dyes/Labels

Fluorescent labels which can be used in determining the percentage of an immune cell subtype present in a sample of PBMCs include, but are not limited to, phycoerythrin (PE), fluorescein isothiocyanate (FITC), allophycocyanin (APC), Texas Red (TR, Molecular Probes, Inc.), peridinin chlorophyll complex (PerCp), CY5 (Biological Detection System) and conjugates thereof coupled to PE (e.g., PE/CY5, PE/APC and PE/TR). Fluorescent labels which can be used in determining the proliferation frequency of an immune cell subtype include, but are not limited to, the PKH dyes such as PKH2, PKH26, and PKH67.

In the foregoing discussion, a number of citations from professional journals and patents are included for reference. All such citations are hereby incorporated in their entirety) by reference.

### EXAMPLE 1

Animals

Productive performance of 199 pigs was monitored for a period of approximately seven months. The pigs were a product of three different internal genotypes: AxC, BXC and BXD genotypes. Eighteen boars in total were used in this trial; eight sires had more than ten offspring and the other ten sires had less than ten. The male to female ratio in the population was 1-1.3.

Animal Flow

Pigs were born at farm A, site I. Site I comprised the breeding, gestation, and farrowing units. The piglets were kept with the sow for a lactation period of approximately 19 days. Sows were fed to meet or exceed internal lactation curve guidelines.

Pig flow took place in weekly batches through the farrowing, nursery, and grow-finishing rooms, and the pigs moved to the next stage when they met specific target weights. The target weight for nursery pigs was 4-6.5 kg, for the “on test” period was 31.7 kg, and to be “off test” was 122.31 kg.

All pigs were processed 24 hours after birth. Each pig was weighed individually (Mosdal scale model IQ-plus 390-DC) and identified with button ear tags. Each pig was given a 1 cc mixture of iron dextran (Durvet, Inc) and penicillin (Pfizerpen® G, Pfizer) and 1 cc of gentamicin (Garacin®, Schering Plough) for scours prevention.

At weaning time, around 19 days of age, each pig was individually weighed and color tagged. Pigs were transported to a nursery on site II where they were housed for at least seven weeks. Site II comprises the nurseries, and growing-finishing rooms. There were nine nurseries and pigs were penned by sex and weight for seven weeks and were housed equally distributed with other non-experimental pigs of the same age and product genotype. A total of 15 pigs/pen was targeted. A four-stage nursery program was implemented as well as a rigorous feed budget program. Pigs were vaccinated at 10 and 24 days of age with a Haemophilus parasuis bacterin (Suvaxyn® Respifend® Hps, Fort Dodge Laboratories).

Blood was obtained from each pig at 6-7 weeks of age by puncturing the anterior vena cava, and was collected in two-10 ml tubes containing anticoagulant (EDTA). Blood samples were kept in coolers with ice packs at all times and sent by early overnight delivery for laboratory processing. The time between blood collection and laboratory processing was 20 hours or less. Blood was sampled in eleven batches to achieve manageable numbers for laboratory processing and a maximum of 25 blood samples were targeted per bleeding session.

At nine weeks of age, pigs were weighed and animals that reached the target weight of 31.7 kg were put “on test” by moving them out of the nurseries into the growing/finishing rooms. Two to four nursery rooms were used to fill one growing/finishing room. Pigs were housed with other non-selected pigs of the same age and genotype. Selected pigs were equally represented in each pen. 60 barrows and 60 gilts were distributed over eight pens within a room in the finishing building. Six rooms of pens were available to house a total of 720 pigs. Pigs were housed with a minimum variation on temperature in all rooms (21.6° C. high and 19.4° C. low) and fed with a four-phase corn-soy based diet program.

Each pig was individually weighed on test (True-Test model 700 scale). In addition, live body measurements were taken, including back fat at the first rib, last rib (back fat), last vertebrae, and loin depth with a real time ultrasound (Aloka model SSD-500 V). Each pig was weighed every two weeks until the pigs reached the target off-test weight 122.3 kg.

At the end of the off-test period, pigs were transported and slaughtered at Swift slaughter facilities (Louisville, Ky.). Carcass traits collected there included hot carcass weight, fat-o-meter back fat, fat-o-meter loin depth and percent lean.

Farm Health Status

Monthly veterinary visits took place in Farm A for the duration of the trial. A veterinarian assessed the presence of clinical disease and mortality in the nursery and growing/finishing units. Farm A was considered to have a low to medium level of Salmonella exposure and to be Mycoplasma hyopneumoniae positive by historical serological studies. The farm was constantly monitored for detection of Salmonella antibodies using the Danish Mixed ELISA at Iowa State University. Results of Salmonella monitoring (ten finishing pigs/month) indicated that there was no active seroconversion during the present study, as seroconversion for Salmonella had occurred in previous years. No clinical signs of Salmonellosis were observed. Mycoplasma hyopneumoniae serology was evaluated yearly. During the last month of this study, 3/10 finishing pigs had antibodies against M. hyopneumoniae as detected by a Tween-ELISA test at the University of Minnesota Diagnostic Laboratory.

Drug/Feed Additive Program

In the pre-nursery and nursery diets, 200 g/ton of neomycin sulfate and oxytetracyclin (Neo-Terramycin®, Pfizer) were used as part of the routine feed additive program.

Cell Preparation

In the laboratory, peripheral blood mononuclear cells (PBMC) from all 199 pig blood samples were isolated by gradient separation using Lymphocyte separation media (LSM, Cappel, ICN Biomedicals, Ohio) as described elsewhere (Solano-Aguilar et al., 2000). After PBMC isolation, cells were counted using Trypan blue dye exclusion and their live cell concentration adjusted to 5×10⁷ cells/ml. Not all pig samples were used for all the immune characterizations.

Cell Types and Monoclonal Antibodies

Natural killer cells (NK) are cells that possess the ability to kill certain tumor cells and a wide variety of virally infected cells. Such killing is part of natural rather than specific immunity. In pigs, NK cells are identified as CD2+/CD16+ double positive cells using the CD2 and CD16 swine monoclonal antibodies (MAbs) MSA3 and G7, respectively. The CD2 MAb detects T lymphocytes, and the CD16 MAb detects the Fc γ receptor type III (FcγRIII).

Killing of target cells by NK requires that the target cell be pre-coated with specific immunoglobulins (IgG), and the lytic process is called antibody-dependent cell-mediated cytotoxicity. Recognition of bound antibody occurs through a low-affinity receptor for IgG Fc on the leukocyte, the FcγRIII, which is detected in swine with the CD16 MAb.

The SLA (swine Major Histocompatibility Complex) molecules play an important role in antigen recognition. They target specific antigens and therefore the SLA belongs to the specific immune system. There are two classes of MHC molecules, class I and class II. CD8+ cells are mostly cytotoxic T lymphocytes (CTL) and recognize peptide fragments bound to class I MHC molecules on cells that are targets of the lytic action of CTL. These peptides are generally derived from endogenously synthesized proteins such as viral antigens. CD4+ cells are mostly helper lymphocytes and recognize peptides bound to class II MHC molecules on the surface of other cells such as B cells, macrophages, and in pigs in some subsets of activated T cells. Class II associated peptides are usually derived from extracellular microbes and soluble protein antigens. CD4+ and CD8+ lymphocyte subsets can be detected using the 74-12-4 and 76-2-11 MAbs, respectively.

In the human and swine MHC and SLA loci, there are distinctive chromosomal regions such as DP, DQ, and DR. In pigs, the SLA-DQ antibodies target the SLA-II antigen as SLA-DQ total (T), bright or active (B), and dull or less active (D). The SLA-DQ MAb recognizes a monomorphic determinant on the SLA-DQ molecule which is thought to be a common component of the region and not a polymorphic determinant (the SLA-DQ gene expresses polymorphic products).

After isolation of PBMC from whole blood, lymphoid cells were tested under two conditions. First, aliquots of PBMC were directly immunostained and the percentages of immune cell subsets determined by incubating the PBMC with primary swine MAb. Secondly, aliquots of PBMC were cultured with the mitogen Concanavalin A (ConA, Sigma, St. Louis, Mo.) and proliferative response of cell subsets determined using flow cytometry and combined MAb and PKH67 staining (Sigma, St. Louis, Mo.). The monoclonal antibodies and their target cells in the PBMC population are listed in Table 1. All monoclonal antibodies used in this study were specific for swine and the specificity to the target cell has been described elsewhere (Saalmueller et al., 1998 and Haverson et al., 2000). These MAbs were chosen because they target surface markers that are expressed on monocytes and lymphocytes. An anti-swine pan leukocyte marker (CD45) and multiple IgG isotype control MAbs were used as positive and negative controls respectively.

The percentage of cells immunostained with the specific MAbs was determined after labeling cells with secondary monoclonal antibodies coupled with FITC or PE (Southern Biotechnology Associates, Birmingham, Ala.).

TABLE 1
Swine Monoclonal Antibodies and Target Cells In PBMC

| Cell Subset | | |
|---|---|---|
| (or | Monoclonal | |
| combination) | Antibody | Target Cell |
| CD2+/CD16+ | | double positive lymphocytes |
| | | expressing both CD2 and CD16; |
| | | Natural Killer (NK) cells |
| CD16 | G7 | low affinity receptor FcγRIII |
| | | expressed primarily by multiple |
| | | cell subsets: NK, B, |
| | | macrophages |
| CD2 | MSA3 | sheep red blood cell receptor; |
| | | predominantly expressed on T |
| | | and NK cells; dull on B cells |
| CD4+/CD8+ | | double positive T lymphocytes |
| | | expressing both CD4 and CD8; |
| | | normally dull CD8 |
| CD4 | 74-12-4 | T lymphocyte subset that reacts |
| | | with MHC type II antigen and |
| | | foreign antigen on antigen |
| | | presenting cell (APC) |
| CD8 | 76-2-11 | T lymphocyte subset that reacts |
| | | with MHC type I antigen and |
| | | foreign antigen on APC |
| SLA-DQ T | TH-16 | swine lymphocyte class (SLA) II |
| | | antigen; equivalent to mice MHC |
| | | class II I-E antigen; |
| | | T = total PBMC (monocytes and |
| | | lymphocyte) |
| SLA-DQ B | | SLA-II B; detects only SLA-DQ |
| | | bright lymphocytes and/or |
| | | macrophages |
| SLA-DQ D | | SLA-II D; detects SLA-DQ dull, |
| | | or less active, lymphocytes |
| | | and/or macrophages |

Flow Cytometric Analyses

Immunostained PBMC were analyzed using flow cytometry as described elsewhere (Solano-Aguilar et al. 2001). The percentage of stained cells was calculated based on the fluorescence intensity using the IgG isotype background as a control. For certain markers it is important to distinguish between dull (D) and bright (B) immunofluorescence thus, for SLA-DQ, the total (T), D and B populations are reported. The flow cytometric analysis for each sample included 16,000 to 20,000 events.

PKH67 Staining

For proliferation testing, PBMC were washed once in RPMI 1640 medium, followed by filtering through a nylon mesh in a 17×100 ml polypropylene conical tube. The cell pellet was taken up in Diluent C (Sigma, St. Louis, Mo.) to achieve a final suspension of 5×10⁶ cells/ml. This cell suspension was then added to an equal volume of PKH67 dye stock (1.5 L dye/1×10⁷ cells) and incubated for three minutes at room temperature. Cell concentrations were previously established to stain cells homogenously and bright. A volume of heat inactivated, fetal bovine serum equal to the total volume of cells+dye was added to the suspension. The cells were then centrifuged to a pellet three times, washed the first time with RPMI medium with serum and the final time in blastogenic media (RPMI 1640 supplemented with 5% FBS, 1 mM sodium pyruvate, 2 mM L-glutamine, non-essential amino acids, 5.5×10-5 M 2-Mercaptoethanol, 25 mM Hepes buffer), and counted with a hemacytometer. Homogeneity of the staining procedure was checked by flow cytometry on day 0 for an aliquot of these labeled cells.

Proliferation

A total of 8×10⁶ PKH67-labeled cells were plated into individual wells of a 24-well tissue culture plate. Concanavalin A at a final dose of 5 μg/ml was added to the wells at the beginning of the culture period. Control wells in medium without mitogen. The final volume was 2 ml/well. The plates were incubated at 37° C. in 5% CO₂ for three days. The top 1 ml of medium was exchanged with 1 ml of fresh blastogenic medium in wells after 48 hours of culture.

Harvesting Cells For Proliferation Analyses

On day three, the cells were collected with two washes in RPMI medium and counted. A concentration of 1×10⁷ cells/ml was used for differential immunostaining as described elsewhere (Solano-Aguilar et al., 2001). After culture cells were stained with control MAbs or CD4 or CD8 MAbs. Data collected was used to calculate lymphocyte proliferation after ConA exposure as a measurement of lymphocyte function. Proliferation of CD4 positive (CD4+), CD8 positive (CD8+), and PKH67 incorporation (total cell proliferation) were compared between cells cultured with Con A or with media to determine if any cell subset was preferentially increased after stimulation.

Data Analysis of Proliferation of Immune Cell

Flow cytometry data was analyzed with the Proliferation Wizard module in ModFit LT software (Verity Software House, Inc., Topsham, Me.). Cells were gated according to the forward and side scattered signals of the lymphocyte population to exclude debris. The intensity of the non-proliferating (parental) cells was determined by analysis of the sample that had been cultured without mitogen. Each generation of cells should have approximately half the PKH67 dye of the parental cells. Working down from the intensity of the parental generation, the ModFit software deconvolutes the fluorescence intensity histogram with Gaussian distributions centered on the peak at different channel intervals (Givan et al., 1999). Using the data returned by the software for the percentage of cells in each daughter generation at the time of the analysis, the individual frequency of each cell subset (CD4+, CD8+, PKH67+) in the original population that had proliferated (precursor frequency or PF) was calculated. The proliferation index (PI) was calculated as the sum of the cells in all generations divided by the number of original parent cells theoretically present in the non-stimulated population. The PI is a measure of the increase in cell number in the culture over the course of the experiment (three days). Precursor frequencies and proliferation indexes were used are measurements of lymphocyte proliferation and are considered the functional assays in this study.

Testing of Immune Parameters as Predictors Of Growth

The productive parameters evaluated included average daily gain (ADG) at the following production stages: a) on test, b) from birth to weaning, c) birth to off test or lifetime daily gain, d) weaning to off test, and e) weaning to on test. Additional productive parameters such as live body and carcass measurements, feed intake, and feed conversion were also analyzed.

Effects of proliferation assays (PF and PI) and immune phenotypes (percentages of cells expressing CD16, CD2, etc) on production parameters were estimated by including them as regressors in a mixed model analysis.

A mixed model (SAS Proc. MIXED) was used to determine which parameters should be used when estimating the effects on growth traits. A full model was used which included product, sire (nested within product), nursery room, finishing room, sex, and parity of sow, where sire is a random effect and all the others are fixed. Sires were nested within product because of partial confounding. Product was not found significant when tested against sire within product and was therefore excluded from the final model. Nursery room and finishing room are strongly confounded, therefore only nursery room is included in the final model as it makes the most significant contribution.

The predictive effect of immune cell subtype markers on proliferation assays and on productive traits were estimated as well as the predictive effect of the proliferation assays on productive traits. The final models for each of these are shown in Table 2.

TABLE 2
Parameter Models

| | |
|---|---|
| Proliferation | sire (product) + nursery room + sex |
| Assays | |
| Immune Markers | sire (product) + nursery room + sex + |
| | parity |
| Live/Carcass | sire (product) + nursery room + sex + |
| Traits | parity + |
| | weight at time of measurement |
| Feed | sire (product) + nursery room + parity |
| Parameters | |

Results

The data analyzed by flow cytometry with the ModFit program indicated that 99.37% of cells were stained with PKH67 demonstrating the high efficiency of the procedure. The results obtained with the CD45 panleukocyte MAb, indicated that an average of 92.04% of the cells isolated were recognized by the CD45 monoclonal antibody as leukocytes, which indicated an appropriate lymphoid gate. Background staining was controlled by the use of IgG isotype controls. All MAb used showed higher values than the background controls indicating that the detection system was effective.

The proportion of the immune marker detected in the blood is given for a typical pig (pig 1867): CD4 (14.92%), CD8 (48.24%), CD4/CD8 (8.32%) SWC3 (4.12%), SLADQ T (46.4%) SLADQB (16.34%), SLADQ D (30.06%), CD16 (20.16%) CD2 (63.32%), CD2/CD16 (18.98%), CD21 (7.26%). The percentages for the controls were: IgGa/Ig2b (0.4/0%), Ig2a/IgG1 (0.22/0.06, CD45 (98.38). As expected, some immune cell subsets like CD8, CD16, CD2/CD16 were relatively abundant. The relative percentages of other markers such as SWC3 and CD21, were less common in the circulating blood but yet several times above the background level.

The average weaning weight was 6.4 kg+0.16, and the average weaning age was 19.4 days±0.2. At weaning, 76% of the piglets fell within the 9.0-14.5 pounds weight range; from the rest, 1% of the pigs fell below a weight of <8.9 pounds and 23% were >14.6 pounds. The average initial on test weight was 33.3 kg±0.5 and the average on test age was 70.8 days±0.6. Average off test weight was 123.1 kg±0.7 and average off test age was 166.8 days±0.9.

These findings were observed under a pig flow of pigs initiating production stages with similar weights rather than similar ages. Standardizing the pig flow for weight was considered important to compare performance in pigs where ADG and carcass evaluation are the traits measured because pig weight will influence daily gain. Similarly, the association analysis for carcass traits took into account the weight of the animal at the time of measurement.

Results of the linear model analysis indicated that overall, the proliferation assays were influenced by the sire and sex; the gain traits were influenced by the sire, the nursery and finishing rooms, sex, and sow parity. Live and carcass measurements were influenced by the product, sire, sex, and sow parity.

After accounting for the various sources of variation, significant associations were found between immune markers and productive performance. These associations were found at the specific production stage that covered the period when the blood sample was collected (nursery) but they were also associated to other production stages suggesting that these markers may predict productive performance through the entire productive life of a pig.

Lymphocyte Proliferation

Immune cell subtype markers were found to be good predictors of lymphocyte proliferation. The total cell proliferation frequency (PKH67 PF) was influenced by the percentage of CD4+ (p=0.09) and SLA-DQT+ cells (p=0.009). The total cell proliferation index (PKH67 PI) was significantly influenced by the percentage of SLA-DQB+ cells (p=0.009). Lastly, the proliferation frequency of CD8+ cells was influenced by the percentage of CD4+/CD8+ cells (p=0.07), CD8+ (p=0.03), and SLA-DQD+ (p=0.06).

Daily Gain

Immune cell subtype markers predicted ADG. Three phenotypes were significantly associated with ADG during the life time of the pig including CD16+ cells (p=0.02), CD2+/CD16+ cells (p=0.0053), CD8+ cells (p=0.04) (Table 3). One functional assay was associated with ADG during the lifetime of the pig: CD4 PF (p=0.08). These four phenotypes were negatively correlated with growth, higher percentages of marker were associated with lower ADG.

Another way to look at the influence of markers on growth is comparing the tails or extremes. It must be stated however that the regressor model as used above is a better indicator than looking at the extremes since it includes all the data. Nevertheless, to assess the impact of frequency of CD2+/CD16+ cells on lifetime ADG, weight gain and the average number of days to market was compared between the 30 pigs showing the highest percentages of these cell subsets and 30 pigs showing the lowest percentage. Weight gain was calculated multiplying ADG by the average number of days necessary to reach off test weight (average for all pigs was 166.8±0.9 days). The average number of days to market was calculated by dividing the target off test weight of 116 kg by the ADG of the top versus the bottom 30 pigs.

The results indicated that an increase of 1% in CD2+/CD16+ cells is predicted to create a reduction of 0.0018 kg of lifetime ADG. So the bottom 30 responders had an average of 5.4% for CD2+/CD16+ cells and the top 30 have an average of 33.1%. This means the predicted difference in lifetime gain is (33.1-5.4)×0.0018=0.0498 kg/day. For 167 days the difference between high and low % of CD2+/CD16+ cells was about 8.32 kg.

Pigs that have a higher proportion of double positive cells had an ADG of 0.7556 kg, so to reach the target off test weight of 116 kg they required in average 156 days. Pigs expressing a high proportion of CD2+/CD16+ cells required 166 days to reach the same target weight making a difference of ten days between groups.

Some phenotypic markers and proliferative responses were significantly associated with ADG at specific production stages but not to the entire productive life of the pig. Those included: CD4+ cells were associated to ADG from birth to weaning (p=0.04), SLA-DQB+cells were associated with ADG from weaning to on test (p=0.08), and SWC3+ cells were associated with ADG on test (p=0.07) and from weaning to on test (p=0.09). All the associations had a positive correlation, the higher the percentage of cells better ADG (Tables 3 and 4).

Carcass Measurements

Significant associations between live body measurements and cell subtype markers and a proliferation assay were found. Those associations included CD4+/CD8+ cells and last vertebrae at on test (p=0.015), CD8 PF and first rib at off test (p=0.08), and SLA-DQD and backfat at on test (p=0.08) (Table 4). Those associations did not remain significant when the carcass traits of the same group of animals were evaluated at the slaughterhouse.

The proportion of cells expressing the swine leukocyte antigens (SLA) class II, or SLA-DQ, markers significantly influenced important carcass traits. The proportion of SLA-DQB+ and SLA-DQT+cells were associated with hot carcass weight (p=0.04). The correlation was positive, higher proportion of SLA-DQB and T positive cells was associated with greater hot carcass weight (Table 4).

Feed Intake and Efficiency

Feed intake was significantly associated with the proportion of SLA-DQD+cells (p=0.04), and SLA-DQT+cells (p=0.06). The SLA-DQ markers were also associated with feed conversion. SLA-DQB+ (p=0.05), SLA-DQD+ (p=0.06), and SLA-DQT (p=0.015) were significantly associated with feed conversion. Pigs with a higher percentage of SLA-DQ positive cells showed better feed conversion. High CD8 PF was associated with worse feed conversion (p=0.09).

No associations were found between production parameters and CD2+, CD21+, CD4 PI, CD8 PI, PKH67 PI, or PKH67 PF.

Overall, the more relevant results from the economic point of view are the significant associations between immune phenotypes and ADG during the pig lifetime, the carcass traits, and feed conversion. Consequently, the biological aspects of the immune cells or cell subsets targeted with the antibodies involved in those key associations will be discussed.

Significant associations were found between CD2+/CD16+ cells and gain traits during the entire productive life of the pigs. These double positive cells are most likely NK cells. NK cells possess the ability to kill certain tumor cells and a wide variety of virally infected cells. Such killing is not induced by specific antigen and is thus part of natural rather than specific immunity. NK cells are capable of lysing various target cells. The level of NK-mediated cytotoxicity is typically not regulated by the antigen but by cytokines and hormones like interleukin-2, interferons (IFN), prolactin, and growth hormone. NK cells also secrete IFN-gamma which activates monocytes to develop into macrophages.

Significant associations were found between CD16+ cells and gain traits during the entire productive life of the pigs. In many cases, killing of target cells by NK requires that the target cell be pre-coated with specific IgG, and the lytic process is called antibody-dependent cell-mediated cytotoxicity. Recognition of bound antibody occurs through a low-affinity receptor for IgG Fc on the leukocyte, called Fc γ receptor III (FcγRIII) which is detected by the CD16 MAb.

Although no absolute marker exists to identify NK cells in swine the authors believe that from the monoclonal antibodies available today, the combination of the CD2/CD16 antibodies seem to be the most appropriate. CD2 expression in human occurs on thymic cells, peripheral T cells, and natural killer cells. It is also expressed on 50% of thymic B cells and expression on mature B cells is controversial. The CD16 detects the FcγRIII. For swine a subset of NK cells are considered to be CD2+/CD16+. The CD16 cell subset is mainly expressed by T and NK cells but B cells may also express it. B and NK cells are CD3 negative. NK cells in pig are divided between CD8- and CD8 dull cells. The role of the B cells as a source of CD16+ cell subsets in this study may not be significant since there were no associations between the B cell antigen (CD21) and ADG.

CD8+ cells are mostly cytotoxic T lymphocytes (CTL) and recognize peptide fragments bound to class I MHC molecules on cells that are targets of the lytic action of CTL. These peptides are generally derived from endogenously synthesized proteins such as viral antigens. MHC class I antigens were not targeted in this study.

CD4+ lymphocytes are mostly helper cells and recognize peptides bound to class II MHC molecules on the surface of other cells such as B cells, macrophages, and in pigs in some subsets of activated T cells. Class II associated peptides are usually derived from extracellular microbes and soluble protein antigens.

The proportion of SLA-DQ+ cells showed significant associations with carcass traits and feed conversion. The SLA or swine MHC molecules play an important role in antigen recognition. The SLA-DQ swine MAb targets the SLA class II antigen, which is thought to be equivalent to the murine MHC class II locus, I-E, and human HLA-DQ. At the gene level, the DQ region comprises loci for alpha and beta chains; SLA-DQ is usually a heterodimer of one alpha and one beta chain which is the surface expressed product detected by the antibody. The SLA-DQ monoclonal antibody recognizes a monomorphic determinant on the SLA-DQ molecule which is thought to be a conserved sequence of one of the genes in the SLA-DQ genes.

The SWC3 MAb was associated to ADG in limited production stages. SWC3 targets mononuclear cells. Mononuclear cells represent a cell population that is critical in natural immunity, and also plays a central role in specific acquired immunity. Some functions of the monocyte are phagocytosis of foreign particles, production of mediators to kill microbes and control the spread of infections, production of cytokines and growth factors, antigen-presenting cell function, and promotion of T cell activation.

High lymphocyte proliferation had a detrimental effect on production parameters such ADG and feed conversion. It has been reported that under acute Salmonella challenge, lymphocyte proliferation is a good indicator of Salmonella resistance and growth. In that study higher proliferative responses of blood lymphocytes prior to challenge were associated with decreased disease resistance and growth under experimental conditions (Van Diemen et al., submitted). In the present study, high proliferative responses were associated with detrimental effects on production parameters including growth which could possibly be associated with resistance to subclinical diseases. Most importantly, this detrimental effect was observed in a commercial setting in a herd with no particular acute disease and it could be an indicator of robustness.

Microbial exposure occurs in any environment that is not completely sterile. Microbial exposure certainly occurs in the cleanest commercial operations. Pig exposure to specific pathogenic agents was not assessed in this study but the association results (ADG) point to the involvement of key immune cells that act in response to viral (NK cells, CD8+ cells) and bacterial infections (CD4+, SLA-DQ+, monocytes). This seems to be relevant even in an environment where there are not evident clinical signs of viral infectious such in the case of farm A.

Several factors could influence the percentages of immune markers present in blood, one of them is vaccination. Vaccination with a Haemophilus parasuis bacterin was administered at days 10 and 24 days of age at the pre-nursery and nursery stages. The second vaccination was given at least three weeks before blood was collected for cell characterization. Because the time lapse after the second vaccination was at least three weeks before blood samples were collected for immunostaining, it is speculated that vaccination did not have a significant impact on the immune cell populations.

Veterinary reports indicated that in farm A, site II some pigs showed clinical disease in the nurseries during the study period. These signs included occasional sick looking pigs, that did not develop well, and coughing. Overall nursery and growing/finishing mortalities were 1-2% and 2% respectively. These percentages are within normal mortality rates in commercial operations. Therefore, 98% of the pigs whose immune cell were tested, reached the end of the experiment. Only 4/199 of the experimental pigs did not survive until the end of the trial.

Management practices at farm A including sex penning, feeding protocols, vaccination, pig density, are similar to many commercial operations. Likewise, the degree of exposure to Salmonella and mycoplasmas is common in many swine operations. Therefore conclusions drawn from this study could be extrapolated to similar operations. The immune cells targeted in this study have conserved functions in other animal species and these cell subtype markers can potentially be used in a broader spectrum of animal hosts.

TABLE 3
Association Between Immune Cell Subtypes (CD16, CD2/CD16,
CD4, CD8) and Proliferative Responses, Average Daily
Gain, Carcass Traits, and Feed Parameters

| Dependent | Number of | Regression | |
|---|---|---|---|
| trait | observations | estimate (se) | p value |
| Effect of | | | |
| CD16 | | | |
| ADG on test | 136 | −0.004 (0.0023) | 0.095¹ |
| ADG from | 139 | −0.002 (0.0009) | 0.03¹ |
| birth to | | | |
| weaning | | | |
| lifetime ADG | 136 | −0.003 (0.0014) | 0.02¹ |
| ADG weaning | 136 | −0.003 (0.0014) | 0.03¹ |
| to off test | | | |
| ADG weaning | 139 | −0.006 (0.0019) | 0.0013¹ |
| to on test | | | |
| Effect of | | | |
| CD2/CD16 | | | |
| ADG on test | 136 | −0.005 (0.0023) | 0.03¹ |
| ADG from | 139 | −0.002 (0.0009) | 0.011¹ |
| birth to | | | |
| weaning | | | |
| lifetime ADG | 136 | −0.004 (0.0014) | 0.0053¹ |
| ADG weaning | 136 | −0.004 (0.0014) | 0.01¹ |
| to off test | | | |
| ADG weaning | 139 | −0.006 (0.0019) | 0.003¹ |
| to on test | | | |
| Effect of | | | |
| CD8 | | | |
| ADG on test | 137 | −0.002 (0.0015) | 0.021 |
| ADG from | 140 | −0.001 (0.0006) | 0.2 |
| birth to | | | |
| weaning | | | |
| lifetime ADG | 137 | −0.002 (0.009) | 0.04¹ |
| ADG weaning | 137 | −0.002 (0.009) | 0.05¹ |
| to off test | | | |
| ADG weaning | 140 | −0.004 (0.0012) | 0.005¹ |
| to on test | | | |
| Effect of | | | |
| CD4 PF | | | |
| ADG on test | 118 | −0.099 (0.0932) | 0.29 |
| ADG from | 120 | −0.013 (0.0494) | 0.8 |
| birth to | | | |
| weaning | | | |
| lifetime ADG | 118 | −0.109 (0.0624) | 0.08¹ |
| ADG weaning | 120 | −0.105 (0.0605) | 0.09¹ |
| to off test | | | |
| ADG weaning | 120 | −0.0141 (0.0818) | 0.09¹ |
| to on test | | | |

¹significant at p < 0.01 level

TABLE 4
Effect of SLA-DQ Subtypes on Carcass
Traits and Feed Parameters

| Dependent | Number of | Regression | |
|---|---|---|---|
| trait | observations | estimate (se) | p value |
| Effect of | | | |
| SLA-DQB | | | |
| first rib at | 139 | −0.0213 (0.0164) | 0.2 |
| on test | | | |
| first rib at | 136 | −0.001 (0.0462) | 0.98 |
| off test | | | |
| backfat at | 139 | −0.005 (0.0122) | 0.69 |
| on test | | | |
| backfat at | 136 | −0.019 (0.0333) | 0.57 |
| off test | | | |
| loin depth | 139 | −0.071 (0.3346) | 0.83 |
| at on test | | | |
| loin depth | 136 | −0.0417 (0.055) | 0.45 |
| at off test | | | |
| last | 139 | −0.0004 (0.0195) | 0.98 |
| vertebrae at | | | |
| on test | | | |
| last | 136 | −0.0059 (0.0258) | 0.82 |
| vertebrae at | | | |
| off test | | | |
| hot carcass | 131 | −0.3587 (0.1753) | 0.04¹ |
| weight | | | |
| fat-o-meter | 131 | −0.003 (0.0347) | 0.93 |
| backfat | | | |
| Fat-o-meter | 131 | −0.0509 (0.0792) | 0.52 |
| loin depth | | | |
| lean | 131 | −0.0078 (0.0249) | 0.76 |
| percentage | | | |
| feed intake | 105 | 0.0048 (0.0043) | 0.27 |
| feed | 105 | 0.0091 (0.0046) | 0.05¹ |
| conversion | | | |
| Effect of | | | |
| SLA-DQD | | | |
| first rib at | 139 | 0.0034 (0.0192) | 0.86 |
| on test | | | |
| first rib at | 136 | 0.036 (0.0536) | 0.51 |
| off test | | | |
| backfat at | 139 | −0.025 (0.014) | 0.08¹ |
| on test | | | |
| backfat at | 136 | 0.0130 (0.0388) | 0.74 |
| off test | | | |
| loin depth | 139 | 0.3441 (0.3877) | 0.38 |
| at on test | | | |
| loin depth | 136 | 0.0615 (0.0638) | 0.34 |
| at off test | | | |
| last | 139 | 0.0091 (0.0226) | 0.69 |
| vertebrae at | | | |
| on test | | | |
| last | 136 | 0.007 (0.0301) | 0.82 |
| vertebrae at | | | |
| off test | | | |
| hot carcass | 131 | 0.1956 (0.2101) | 0.35 |
| weight | | | |
| fat-o-meter | 131 | 0.0164 (0.0402) | 0.68 |
| backfat | | | |
| Fat-o-meter | 131 | 0.0510 (0.0919) | 0.58 |
| loin depth | | | |
| lean | 131 | 0.004 (0.0288) | 0.9 |
| percentage | | | |
| feed intake | 105 | 0.0109 (0.0053) | 0.04¹ |
| feed | 105 | 0.0111 (0.0058) | 0.06¹ |
| conversion | | | |
| Effect of | | | |
| SLA-DQT | | | |
| first rib at | 139 | 0.0122 (0.0118) | 0.3 |
| on test | | | |
| first rib at | 136 | −0.014 (0.0329) | 0.67 |
| off test | | | |
| backfat at | 139 | −0.012 (0.0087) | 0.17 |
| on test | | | |
| backfat at | 136 | −0.005 (0.0238) | 0.85 |
| off test | | | |
| loin depth | 139 | 0.0942 (0.2394) | 0.69 |
| at on test | | | |
| loin depth | 136 | 0.0442 (0.039) | 0.26 |
| at off test | | | |
| last | 139 | 0.0037 (0.0139) | 0.79 |
| vertebrae at | | | |
| on test | | | |
| last | 136 | 0.0005 (0.0184) | 0.98 |
| vertebrae at | | | |
| off test | | | |
| hot carcass | 131 | 0.2616 (0.1269) | 0.04¹ |
| weight | | | |
| fat-o-meter | 131 | 0.0048 (0.0251) | 0.85 |
| backfat | | | |
| fat-o-meter | 131 | 0.0466 (0.0573) | 0.42 |
| loin depth | | | |
| lean | 131 | 0.0026 (0.018) | 0.89 |
| percentage | | | |
| feed intake | 105 | 0.0057 (0.0029) | 0.06¹ |
| feed | 105 | 0.0078 (0.0031) | 0.015¹ |
| conversion | | | |

¹significant at p < 0.01 level

### EXAMPLE 2

The size of the pig population studied was increased to an additional 286 pigs. The goal was to collect more data to confirm the associations between the immune phenotypes (CD16, CD2/CD16, CD8) and productive performance (growth). The pigs included in this study originated from 2 different farms, one of the farms was the same from Example 1. The management of the animals, the productive performance recording, and the measurement of immunological traits was similar to the one described in Example 1. Similarly, the statistical analysis was performed in an identical fashion to the one described in Example 1. The association analysis reported for the 1st year included 139 animals. The 1^{st} and 2^{nd} year results are presented together for total of 425 animals. See Table 5 below.

TABLE 5
Effect of immune phenotypes in average daily gain (ADG). The statistical
model: sire (product) + nursery room (year) + sex + parity
was used. For a dataset of this size results with a p value > 0.1
are considered significant. In bold years 1 and 2 are merged together.
Non-bolded results are from year 1 only.

| | | | |
|---|---|---|---|
| | Number of | Regression | P |
| Growth parameter | observations | estimate (se) | value |

CD16

| | | | |
|---|---|---|---|
| ADG on test | 425 | −.829 (.5170) | 0.11 |
| ADG from birth | 139 | −.002 (.0009) | .003 |
| to weaning | | | |
| Lifetime ADG | 425 | −.580 (.3451) | 0.09 |
| ADG weaning to | 225 | −.002 (.0013) | 0.13 |
| off test | | | |
| ADG weaning to | 139 | −.006 (.0019) | .0012 |
| on test | | | |

CD2/CD16

| | | | |
|---|---|---|---|
| ADG on test | 425 | −.572 (.5387) | 0.29 |
| ADG from birth | 139 | −.002 (.0009) | 0.011 |
| to weaning | | | |
| Lifetime ADG | 425 | −.339 (.3598) | 0.35 |
| ADG weaning to | 225 | −.002 (.0014) | 0.096 |
| off test | | | |
| ADG weaning to | 139 | −.006 (.0019) | 0.003 |
| on test | | | |

CD8

| | | | |
|---|---|---|---|
| ADG on test | 425 | −.134 (.3288) | 0.68 |
| ADG from birth | 139 | −.001 (.0006) | 0.19 |
| to weaning | | | |
| Lifetime ADG | 425 | −.068 (.2212) | 0.76 |
| ADG weaning to | 225 | −.002 (.0008) | 0.002 |
| off test | | | |
| ADG weaning to | 139 | −.004 (.0012) | 0.0045 |
| on test | | | |

From Table 5 above it is important to point out the following:

These additional results provide evidence that the percentage of CD16 positive cells is associated with growth during the entire productive life of the pigs (p>0.09).

Additionally, these results provide evidence that the percentage of CD2+/CD16+ cells is associated with growth. In this case the associations were significant for ADG from weaning to off test (p>0.096) but not necessarily during the lifetime ADG.

Furthermore, these results provide evidence that the percentage of CD8 positive cells is associated with growth in pigs. In this case the associations were significant for ADG from weaning to off test (p>0.002) but not necessarily during the entire lifetime ADG.

### REFERENCES

Bevan et al., Science 264:796-7 (1994).

Cole et al., MONOCLONAL ANTIBODIES AND CANCER THERAPY, Alan R. Liss, Inc., pp. 77-96 (1985).

Dulbecco et al., Virology 8:396 (1959).

Givan et al., J. Immunol. Meth. 230:99-112 (1999).

Haverson et al., Vet. Immunol. Immunopathol. 80:5-23 (2001).

Kohler and Milstein, Nature 256:495-7 (1975).

Kozbor et al., Immunol. Today 4:72 (1983).

Marks et al., J. Biol. Chem. 16007-10 (1992).

Mosier, Science 158:1573-5 (1967).

Miedema and Melief, Immunol. Today 6:258-9 (1983).

Robertson, Nature 301:114 (1983).

Saalmueller et al., Vet. Immunol. Immunopathol. 60:207-28 (1998).

Solano-Aguilar et al., J. Immunol. Meth. 241:185-99 (2000).

Solano-Aguilar et al., Int'l. J. Parasitol. 31:187-95 (2001).

Tizard, IMMUNOLOGY: AN INTRODUCTION, Saunders, Philadelphia (1988).

Van Diemen et al., submitted to the Journal of Immunology and Immunopathology, 2002.

Zola et al., in MONOCLONAL HYBRIDOMA ANTIBODIES: TECHNIQUES AND APPLICATIONS, Hurell ed., CRC Press, pp. 51-2 (1982).

One of ordinary skill in the art, with the aid of the present disclosure, can affect various changes, substitutions of equivalents and other alterations to the methods and compositions herein set forth, in order to practice this invention. Therefore, the protection granted by letters patent should not be limited except by the language of the claims as set forth below.

What is claimed is:

1. A method for selecting for robustness among two or more animals, the method comprising the steps of:
providing two or more animals of the same species;
determining in each animal the quantity of CD16 antigen-expressing cells; and
selecting the animal with the lowest quantity of CD16 antigen-expressing cells,
thereby selecting for robustness among two or more animals.

2. A method for selecting for robustness among two or more animals, the method comprising the steps of:
providing two or more animals of the same species;
determining in each animal the quantity of CD16 and CD2 double-positive antigen-expressing cells; and
selecting the animal with the lowest quantity of CD16 and CD2 double-positive antigen-expressing cells,
thereby selecting for robustness among two or more animals.

3. A method for selecting for robustness among two or more animals, the method comprising the steps of:
providing two or more animals of the same species;
determining in each animal the quantity of CD8 antigen-expressing cells; and
selecting the animal with the lowest quantity of CD8 antigen-expressing cells,
thereby selecting for robustness among two or more animals.

4. A method for selecting for robustness among two or more animals, the method comprising the steps of:
providing two or more animals of the same species;
determining in each animal the quantity of MHC-DQ antigen-expressing cells; and
selecting the animal with the highest quantity of MHC-DQ antigen-expressing cells,
thereby selecting for robustness among two or more animals.

5. A method for selecting for robustness among two or more animals, the method comprising the steps of:
providing two or more animals of the same species;
determining in each animal the quantity of cells expressing an antigen that is targeted by MHC-DQ antibodies as MHC-DQB; and
selecting the animal with the highest quantity of cells expressing an antigen that is targeted by MHC-DQ antibodies as MHC-DQB,
thereby selecting for robustness among two or more animals.

6. A method for selecting for robustness among two or more animals, the method comprising the steps of:
providing two or more animals of the same species;
determining in each animal the quantity of cells expressing an antigen that is targeted by MHC-DQ antibodies as MHC-DQD; and
selecting the animal with the highest quantity of cells expressing an antigen that is targeted by MHC-DQ antibodies as MHC-DQD,
thereby selecting for robustness among two or more animals.

7. A method for selecting for robustness among two or more animals, the method comprising the steps of:
providing two or more animals of the same species;
determining in each animal the proliferation frequency of CD4 antigen-expressing cells; and
selecting the animal with the lowest proliferation frequency of CD4 antigen-expressing cells,
thereby selecting for robustness among two or more animals.

8. The method of claim 1, wherein the species is selected from the group consisting of Bos taurus, Sus scrofa, Ovis aries, Bison bison, Babalus babalus, Gallus domesticus, Meleagrus gallopavo, Anas rubripes, and Branta canadensis.

9. The method of claim 2, wherein the species is selected from the group consisting of Bos taurus, Sus scrofa, Ovis aries, Bison bison, Babalus babalus, Gallus domesticus, Meleagrus gallopavo, Anas rubripes, and Branta canadensis.

10. The method of claim 3, wherein the species is selected from the group consisting of Bos taurus, Sus scrofa, Ovis aries, Bison bison, Babalus babalus, Gallus domesticus, Meleagrus gallopavo, Anas rubripes, and Branta canadensis.

11. The method of claim 4, wherein the species is selected from the group consisting of Bos taurus, Sus scrofa, Ovis aries, Bison bison, Babalus babalus, Gallus domesticus, Meleagrus gallopavo, Anas rubripes, and Branta canadensis.

12. The method of claim 5, wherein the species is selected from the group consisting of Bos taurus, Sus scrofa, Ovis aries, Bison bison, Babalus babalus, Gallus domesticus, Meleagrus gallopavo, Anas rubripes, and Branta canadensis.

13. The method of claim 6, wherein the species is selected from the group consisting of Bos taurus, Sus scrofa, Ovis aries, Bison bison, Babalus babalus, Gallus domesticus, Meleagrus gallopavo, Anas rubripes, and Branta canadensis.

14. The method of claim 7, wherein the species is selected from the group consisting of Bos taurus, Sus scrofa, Ovis aries, Bison bison, Babalus babalus, Gallus domesticus, Meleagrus gallopavo, Anas rubripes, and Branta canadensis.

15. A method for selecting for robustness among two or more animals, the method comprising the steps of:
providing two or more animals of the same species;
determining in each animal the quantities of CD16 antigen-expressing cells, CD16 and CD2 double-positive antigen-expressing cells, CD8 antigen-expressing cells, MHC-DQ antigen-expressing cells, cells expressing an antigen that is targeted by MHC-DQ antibodies as MHC-DQB, and cells expressing an antigen that is targeted by MHC-DQ antibodies as MHC-DQD;
determining in each animal the proliferation frequency of CD4 antigen-expressing cells; and
selecting the animal with the lowest quantity of CD16 antigen-expressing cells, the lowest quantity of CD16 and CD2 double-positive antigen-expressing cells, the lowest quantity of CD8 antigen-expressing cells, the highest quantity of MHC-DQ antigen-expressing cells, the highest quantity of cells expressing an antigen targeted by MHC-DQ antibodies as MHC-DQB, the highest quantity of cells expressing an antigen targeted by MHC-DQ antibodies as MHC-DQD, and the lowest proliferation frequency of CD4 antigen-expressing cells,
thereby selecting for robustness among two or more animals.

16. The method of claim 15, wherein the species is selected from the group consisting of Bos taurus, Sus scrofa, Ovis aries, Bison bison, Babalus babalus, Gallus domesticus, Meleagrus gallopavo, Anas rubripes, and Branta canadensis.

17. A method for selecting for robustness among two or more animals, the method comprising the steps of:
providing two or more animals of the same species;
determining in each animal the quantity of CD16 antigen-expressing cells;
determining a statistically significant association between an animal's quantity of CD16 antigen-expressing cells and robustness; and
selecting for the animal in order to improve robustness based on the association.

18. A method for selecting for robustness among two or more animals, the method comprising the steps of:
providing two or more animals of the same species;
determining in each animal the quantity of CD16 and CD2 double-positive antigen-expressing cells;
determining a statistically significant association between an animal's quantity of CD16 and CD2 double-positive antigen-expressing cells and robustness; and
selecting for the animal in order to improve robustness based on the association.

19. A method for selecting for robustness among two or more animals, the method comprising the steps of:
providing two or more animals of the same species;
determining in each animal the quantity of CD8 antigen-expressing cells;
determining a statistically significant association between an animal's quantity of CD8 antigen-expressing cells and robustness; and
selecting for the animal in order to improve robustness based on the association.

20. A method for selecting for robustness among two or more animals, the method comprising the steps of:
providing two or more animals of the same species;
determining in each animal the quantity of MHC-DQ antigen-expressing cells;
determining a statistically significant association between an animal's quantity of MHC-DQ antigen-expressing cells and robustness; and
selecting for the animal in order to improve robustness based on the association.

21. A method for selecting for robustness among two or more animals, the method comprising the steps of:
providing two or more animals of the same species;
determining in each animal the quantity of cells expressing an antigen that is targeted by MHC-DQ antibodies as MHC-DQB;
determining a statistically significant association between an animal's quantity of cells expressing an antigen that is targeted by MHC-DQ antibodies as MHC-DQB and robustness; and
selecting for the animal in order to improve robustness based on the association.

22. A method for selecting for robustness among two or more animals, the method comprising the steps of:
providing two or more animals of the same species;
determining in each animal the quantity of cells expressing an antigen that is targeted by MHC-DQ antibodies as MHC-DQD;
determining a statistically significant association between an animal's quantity of cells expressing an antigen that is targeted by MHC-DQ antibodies as MHC-DQD; and
selecting for the animal in order to improve robustness based on the association.

23. A method for selecting for robustness among two or more animals, the method comprising the steps of:
providing two or more animals of the same species;
determining in each animal the proliferation frequency of CD4 antigen-expressing cells;
determining a statistically significant association between an animal's proliferation frequency of CD4 antigen-expressing cells and robustness; and
selecting for the animal in order to improve robustness based on the association.

24. The method of claim 17 wherein the species is selected from the group consisting of Bos taurus, Sus scrofa, Ovis aries, Bison bison, Babalus babalus, Gallus domesticus, Meleagrus gallopavo, Anas rubripes, and Branta canadensis.

25. The method of claim 18 wherein the species is selected from the group consisting of Bos taurus, Sus scrofa, Ovis aries, Bison bison, Babalus babalus, Gallus domesticus, Meleagrus gallopavo, Anas rubripes, and Branta canadensis.

26. The method of claim 19 wherein the species is selected from the group consisting of Bos taurus, Sus scrofa, Ovis aries, Bison bison, Babalus babalus, Gallus domesticus, Meleagrus gallopavo, Anas rubripes, and Branta Canadensis.

27. The method of claim 20 wherein the species is selected from the group consisting of Bos taurus, Sus scrofa, Ovis aries, Bison bison, Babalus babalus, Gallus domesticus, Meleagrus gallopavo, Anas rubripes, and Branta Canadensis.

28. The method of claim 21 wherein the species is selected from the group consisting of Bos taurus, Sus scrofa, Ovis aries, Bison bison, Babalus babalus, Gallus domesticus, Meleagrus gallopavo, Anas rubripes, and Branta Canadensis.

29. The method of claim 22 wherein the species is selected from the group consisting of Bos taurus, Sus scrofa, Ovis aries, Bison bison, Babalus babalus, Gallus domesticus, Meleagrus gallopavo, Anas rubripes, and Branta Canadensis.

30. The method of claim 23 wherein the species is selected from the group consisting of Bos taurus, Sus scrofa, Ovis aries, Bison bison, Babalus babalus, Gallus domesticus, Meleagrus gallopavo, Anas rubripes, and Branta Canadensis.

31. A method for selecting for robustness among two or more animals, the method comprising the steps of:
providing two or more animals of the same species;
obtaining a biological sample from the animals, wherein the sample comprises whole blood;
determining in each animal the quantity of CD16 antigen-expressing cells; and
selecting the animal with the lowest quantity of CD16 antigen-expressing cells,
thereby selecting for robustness among two or more animals.

32. A method for selecting for robustness among two or more animals, the method comprising the steps of:
providing two or more animals of the same species;
obtaining a biological sample from the animals, wherein the sample comprises whole blood;
determining in each animal the quantity of CD16 and CD2 double-positive antigen-expressing cells; and
selecting the animal with the lowest quantity of CD16 and CD2 double-positive antigen-expressing cells,
thereby selecting for robustness among two or more animals.

33. A method for selecting for robustness among two or more animals, the method comprising the steps of:
providing two or more animals of the same species;
obtaining a biological sample from the animals, wherein the sample comprises whole blood;
determining in each animal the quantity of CD8 antigen-expressing cells; and
selecting the animal with the lowest quantity of CD8 antigen-expressing cells,
thereby selecting for robustness among two or more animals.

34. A method for selecting for robustness among two or more animals, the method comprising the steps of:
providing two or more animals of the same species;
obtaining a biological sample from the animals, wherein the sample comprises whole blood;
determining in each animal the quantity of MHC-DQ antigen-expressing cells; and
selecting the animal with the highest quantity of MHC-DQ antigen-expressing cells,
thereby selecting for robustness among two or more animals.

35. A method for selecting for robustness among two or more animals, the method comprising the steps of:
providing two or more animals of the same species;
obtaining a biological sample from the animals, wherein the sample comprises whole blood;
determining in each animal the quantity of cells expressing an antigen that is targeted by MHC-DQ antibodies as MHC-DQB; and
selecting the animal with the highest quantity of cells expressing an antigen that is targeted by MHC-DQ antibodies as MHC-DQB,
thereby selecting for robustness among two or more animals.

36. A method for selecting for robustness among two or more animals, the method comprising the steps of:
providing two or more animals of the same species;
obtaining a biological sample from the animals, wherein the sample comprises whole blood;
determining in each animal the quantity of cells expressing an antigen that is targeted by MHC-DQ antibodies as MHC-DQD; and
selecting the animal with the highest quantity of cells expressing an antigen that is targeted by MHC-DQ antibodies as MHC-DQD,
thereby selecting for robustness among two or more animals.

37. A method for selecting for robustness among two or more animals, the method comprising the steps of:
providing two or more animals of the same species;
obtaining a biological sample from the animals, wherein the sample comprises whole blood;
determining in each animal the proliferation frequency of CD4 antigen-expressing cells; and
selecting the animal with the lowest proliferation frequency of CD4 antigen-expressing cells,
thereby selecting for robustness among two or more animals.

38. The method of claim 31 wherein the species is selected from the group consisting of Bos taurus, Sus scrofa, Ovis aries, Bison bison, Babalus babalus, Gallus domesticus, Meleagrus gallopavo, Anas rubripes, and Branta canadensis.

39. The method of claim 32 wherein the species is selected from the group consisting of Bos taurus, Sus scrofa, Ovis aries, Bison bison, Babalus babalus, Gallus domesticus, Meleagrus gallopavo, Anas rubripes, and Branta canadensis.

40. The method of claim 33 wherein the species is selected from the group consisting of Bos taurus, Sus scrofa, Ovis aries, Bison bison, Babalus babalus, Gallus domesticus, Meleagrus gallopavo, Anas rubripes, and Branta Canadensis.

41. The method of claim 34 wherein the species is selected from the group consisting of Bos taurus, Sus scrofa, Ovis aries, Bison bison, Babalus babalus, Gallus domesticus, Meleagrus gallopavo, Anas rubripes, and Branta Canadensis.

42. The method of claim 35 wherein the species is selected from the group consisting of Bos taurus, Sus scrofa, Ovis aries, Bison bison, Babalus babalus, Gallus domesticus, Meleagrus gallopavo, Anas rubripes, and Branta Canadensis.

43. The method of claim 36 wherein the species is selected from the group consisting of Bos taurus, Sus scrofa, Ovis aries, Bison bison, Babalus babalus, Gallus domesticus, Meleagrus gallopavo, Anas rubripes, and Branta Canadensis.

44. The method of claim 37 wherein the species is selected from the group consisting of Bos taurus, Sus scrofa, Ovis aries, Bison bison, Babalus babalus, Gallus domesticus, Meleagrus gallopavo, Anas rubripes, and Branta Canadensis.

45. A method for selecting for robustness among two or more animals, the method comprising the steps of:
providing two or more animals of the same species;
obtaining a biological sample from the animals, wherein the sample comprises whole blood;
determining in each animal the quantity of CD16 antigen-expressing cells;
determining a statistically significant association between an animal's quantity of CD16 antigen-expressing cells and robustness; and
selecting for the animal in order to improve robustness based on the association.

46. A method for selecting for robustness among two or more animals, the method comprising the steps of:
providing two or more animals of the same species;
obtaining a biological sample from the animals, wherein the sample comprises whole blood;
determining in each animal the quantity of CD16 and CD2 double-positive antigen-expressing cells;
determining a statistically significant association between an animal's quantity of CD16 and CD2 double-positive antigen-expressing cells and robustness; and
selecting for the animal in order to improve robustness based on the association.

47. A method for selecting for robustness among two or more animals, the method comprising the steps of:
providing two or more animals of the same species;
obtaining a biological sample from the animals, wherein the sample comprises whole blood;
determining in each animal the quantity of CD8 antigen-expressing cells;
determining a statistically significant association between an animal's quantity of CD8 antigen-expressing cells and robustness; and
selecting for the animal in order to improve robustness based on the association.

48. A method for selecting for robustness among two or more animals, the method comprising the steps of:
providing two or more animals of the same species;
obtaining a biological sample from the animals, wherein the sample comprises whole blood;
determining in each animal the quantity of MHC-DQ antigen-expressing cells;
determining a statistically significant association between an animal's quantity of MHC-DQ antigen-expressing cells and robustness; and
selecting for the animal in order to improve robustness based on the association.

49. A method for selecting for robustness among two or more animals, the method comprising the steps of:
providing two or more animals of the same species;
obtaining a biological sample from the animals, wherein the sample comprises whole blood;
determining in each animal the quantity of cells expressing an antigen that is targeted by MHC-DQ antibodies as MHC-DQB;
determining a statistically significant association between an animal's quantity of cells expressing an antigen that is targeted by MHC-DQ antibodies as MHC-DQB and robustness; and
selecting for the animal in order to improve robustness based on the association.

50. A method for selecting for robustness among two or more animals, the method comprising the steps of:
providing two or more animals of the same species;
obtaining a biological sample from the animals, wherein the sample comprises whole blood;
determining in each animal the quantity of cells expressing an antigen that is targeted by MHC-DQ antibodies as MHC-DQD;
determining a statistically significant association between an animal's quantity of cells expressing an antigen that is targeted by MHC-DQ antibodies as MHC-DQD; and
selecting for the animal in order to improve robustness based on the association.

51. A method for selecting for robustness among two or more animals, the method comprising the steps of:
providing two or more animals of the same species;
obtaining a biological sample from the animals, wherein the sample comprises whole blood;
determining in each animal the proliferation frequency of CD4 antigen-expressing cells;
determining a statistically significant association between an animal's proliferation frequency of CD4 antigen-expressing cells and robustness; and
selecting for the animal in order to improve robustness based on the association.

52. The method of claim 45 wherein the species is selected from the group consisting of Bos taurus, Sus scrofa, Ovis aries, Bison bison, Babalus babalus, Gallus domesticus, Meleagrus gallopavo, Anas rubripes, and Branta canadensis.

53. The method of claim 46 wherein the species is selected from the group consisting of Bos taurus, Sus scrofa, Ovis aries, Bison bison, Babalus babalus, Gallus domesticus, Meleagrus gallopavo, Anas rubripes, and Branta canadensis.

54. The method of claim 47 wherein the species is selected from the group consisting of Bos taurus, Sus scrofa, Ovis aries, Bison bison, Babalus babalus, Gallus domesticus, Meleagrus gallopavo, Anas rubripes, and Branta Canadensis.

55. The method of claim 48 wherein the species is selected from the group consisting of Bos taurus, Sus scrofa, Ovis aries, Bison bison, Babalus babalus, Gallus domesticus, Meleagrus gallopavo, Anas rubripes, and Branta Canadensis.

56. The method of claim 49 wherein the species is selected from the group consisting of Bos taurus, Sus scrofa, Ovis aries, Bison bison, Babalus babalus, Gallus domesticus, Meleagrus gallopavo, Anas rubripes, and Branta Canadensis.

57. The method of claim 50 wherein the species is selected from the group consisting of Bos taurus, Sus scrofa, Ovis aries, Bison bison, Babalus babalus, Gallus domesticus, Meleagrus gallopavo, Anas rubripes, and Branta Canadensis.

58. The method of claim 51 wherein the species is selected from the group consisting of Bos taurus, Sus scrofa, Ovis aries, Bison bison, Babalus babalus, Gallus domesticus, Meleagrus gallopavo, Anas rubripes, and Branta canadensis.
